(19) United States
(12) Patent Application Publication
(10) Pub. No.: US 2015/0164601 A1
(43) Pub. Date: Jun. 18, 2015

(54) INTERFACE BETWEEN USER AND LAPAROSCOPIC TOOLS

(71) Applicant: Human Extensions Ltd., Netanya (IL)

(72) Inventor: Mordehai SHOLEV, Moshav Amikam (IL)

(21) Appl. No.: 14/632,061

(22) Filed: Feb. 26, 2015

Related U.S. Application Data
(63) Continuation of application No. PCT/IL2013/050731, filed on Aug. 28, 2013.
(60) Provisional application No. 61/694,865, filed on Aug. 30, 2012.

(51) Int. Cl. A61B 19/00

(52) U.S. Cl. CPC A61B 19/26; A61B 2019/261

(57) ABSTRACT

The laparoscopic tool interface comprises a frame attachable to an upper extremity of a user; a controller operatively connected to a laparoscopic tool and arranged for manipulation by a hand of a user; and, a mounting on the frame for the laparoscopic tool; and, wherein operating parts for tool position movements of the laparoscopic tool being separated from the controller and wherein a shaft of said laparoscopic tool not being coaxial with the upper extremity of said user and/or not being coaxial with said controller. The functions of positioning of the laparoscopic tool and operating the tool are separated. Movement of the upper extremity of the user and/or movement of a joint enable positioning of the tool to a preferred point and at the right orientation. The fingers of the user effect operational movement of the tool.

### RELATED APPLICATIONS

This application is a Continuation of PCT Patent Application No. PCT/IL2013/050731 having International filing date of Aug. 28, 2013, which claims the benefit of priority under 35 USC §119(e) of U.S. Provisional Patent Application No. 61/694,865 filed on Aug. 30, 2012. The contents of the above applications are all incorporated by reference as if fully set forth herein in their entirety.

### FIELD OF THE INVENTION

The present invention, in some embodiments thereof, relates to a medical device and, more particularly, but not exclusively, to an interface between the surgeon and the laparoscopic tool.

### BACKGROUND OF THE INVENTION

One of the more frequently used medical procedures is minimally invasive surgical (MIS). Minimally Invasive Surgery is a surgical procedure that typically relies on a small camera and thin tools being introduced through small incisions to perform a procedure that would normally require open surgery with a long incision to provide access for conventional size tools. MIS procedures have the following advantages: reduced trauma, blood-loss, scarring, and post-operative pain; fewer post-operative complications; and, faster recovery times and shorter hospital stays. Some of the problems with MIS procedures are that: the tools are hard to use and to manipulate and there is only a limited degree of freedom of movement of the surgical tool; the workplaces are small and there is limited access room for the tools; surgeons require comprehensive training; and the surgeon has reduced visibility and depth perception at the incision.

These MIS procedures require generally thin tools that are inserted into the body through ports. An aspect of these tools is that the motion of the user, such as a surgeon, is transferred via the tool and directs the motion of a manipulator attached to the tool's tip inside the patient's body. By means of such procedures, it is possible to effect an externally controlled operation within the body without making large incisions. Many types of tools can be used in this manner, ranging from simple scissor-like tools to complex robotic systems.

Awtar (US Published Patent Application 2012/0041450) is a minimal access tool and includes a frame arranged to be attached to an arm of a user. A tool shaft has a proximal end connected to the frame. The tool further includes an input joint having a first end connected to the frame and a second end arranged to receive user input, the input joint including a virtual center-of-rotation (VC) mechanism which provides a center of rotation that generally coincides with a wrist joint of the user. An output joint is connected to the tool shaft distal end, where the output joint is coupled to the input joint via a mechanical transmission connected there between to correlate motion of the input joint to motion of the output joint.

Gotani (U.S. Pat. No. 7,572,253) discloses that in order to rotate and move an arm of a slave which supports a needle-holder, the rotation and movement of a pen-shaped operating section caused by the fingers of the operator's hand are directly transmitted as the rotation and movement of the needle-holder. Sensors for detecting the rotation and movement of the operating section, an X-axis torque sensor, Y-axis torque sensor, Z-axis torque sensor, and a rotation detecting potentiometer are installed thereon. As a result, the rotation and movement of the pen-shaped operating section caused by the fingers of the operator's hand are transmitted as the rotation and movement of the needle-holder in optimum proportions through a computing section. Therefore, when the needle-holder is to be finely moved, a subtle motion of the fingers of the hand can be satisfactorily transmitted. Thereby, it is possible to provide a surgical operation device capable of doing minute surgical operations with ease.

Lee (US Published Patent Application 2008/0255420) discloses a surgical instrument having an instrument shaft having proximal and distal ends, a tool disposed from the distal end of the instrument shaft, a control handle coupled from the proximal end of the instrument shaft, a distal motion member for coupling the distal end of the instrument shaft to the tool, a proximal motion member for coupling the proximal end of the instrument shaft to the handle and actuation means extending between distal and proximal motion members for coupling motion of the proximal motion member to the distal motion member for controlling the positioning of the tool. Rotation control and locking members are also disclosed.

### SUMMARY OF THE INVENTION

An aspect of some embodiments of the invention relates to separating the functions of positioning of the laparoscopic tool and operating the tool. In an exemplary embodiment of the invention, movement of the upper extremity (for example, the forearm and/or hand of the user) and/or movement of a joint enable positioning of the tool to a preferred point and at the right orientation. Other body parts of the user, primarily the fingers, effect operational movement of the tool.

This separation of the functions of positioning and operating the tool is advantageous compared to the way a surgeon currently uses the laparoscopic tools. At present, the surgeon uses his fingers to position the tool and also to operate the tool. This forces the surgeon to work in a non ergonomic manner for long time and to operate the tool with a limitation of finger movement.

According to an aspect of some embodiments of the present invention there is provided a method of using a laparoscopic tool comprising the steps of: attaching a frame to an upper extremity (for example the forearm or hand) of a user; positioning a mounting on said frame for said laparoscopic tool; securing said laparoscopic tool in said mounting; operatively connecting a controller to said laparoscopic tool and arranging said controller for manipulation by a hand of said user; and, initiating movement of said laparoscopic tool by manipulation of said controller and/or by movement of an upper extremity of said user.

According to some embodiments of the invention, manipulation of the controller by the hand of the user effects operation of the laparoscopic tool; and, positioning of the laparoscopic tool is effected by movement of the upper extremity of the user and/or the mounting.

According to some embodiments of the invention, movement of the laparoscopic tool is not caused by movement of the shoulder of the user.

According to some embodiments of the invention, a relaxed position of the laparoscopic device is at an approximate 90 degree relationship to the upper extremity of the user.

According to some embodiments of the invention, a shaft of the laparoscopic tool is maintained in a relative position to the upper extremity of the user.

According to some embodiments of the invention, the shaft of the laparoscopic tool is maintained in a relative position to the upper extremity of the user even when the upper extremity of the user is moved.

According to some embodiments of the invention, a relative position of the shaft of the laparoscopic tool is changed with respect to the upper extremity of the user.

According to some embodiments of the invention, an angle between the shaft of the laparoscopic tool and the upper extremity of the user is changed.

According to some embodiments of the invention, tilting of the shaft of the laparoscopic tool with respect to the mounting is prevented.

According to some embodiments of the invention, rotation of the shaft of the laparoscopic tool in the mounting is prevented.

According to an aspect of some embodiments of the present invention there is provided a laparoscopic tool interface comprising: a frame attachable to an upper extremity of a user; a controller operatively connected to a laparoscopic tool and arranged for manipulation by a hand of a user; and, a mounting on said frame for the laparoscopic tool; and, wherein operating parts for tool position movements of the laparoscopic tool being separated from the controller and wherein a shaft of the laparoscopic tool not being coaxial with the upper extremity of the user.

According to some embodiments of the invention, the mounting comprises a joint between the tool shaft of the laparoscopic tool and the frame.

According to some embodiments of the invention, the laparoscopic tool interface comprises articulating elements for changing an angle between the shaft of the laparoscopic tool and the upper extremity of the user.

According to some embodiments of the invention, the articulating elements comprise a pair of arc shaped tracks mounted on a distal end of the frame, and means for operatively moving the mounting in relation to the arc shaped tracks to facilitate angular displacement of the laparoscopic tool.

According to some embodiments of the invention, the laparoscopic tool interface comprises an articulated link mechanism for supporting the joint on said frame.

According to some embodiments of the invention, the articulated link mechanism maintains the shaft of the laparoscopic tool in a relative position to the upper extremity of the user.

According to some embodiments of the invention, the articulated link mechanism facilitates changing a relative position of the shaft of the laparoscopic tool with respect to the upper extremity of the user.

According to some embodiments of the invention, the joint facilitates a relaxed position of the laparoscopic device to be at an approximate 90 degree relationship to the upper extremity of the user.

According to some embodiments of the invention, the joint maintains the shaft of the laparoscopic tool in a relative position to the upper extremity of the user.

According to some embodiments of the invention, the joint maintains the shaft of the laparoscopic tool in a relative position to the upper extremity of the user even when the upper extremity of the user is moving.

According to some embodiments of the invention, the tool shaft is at an arbitrary angle to the upper extremity of the user.

According to some embodiments of the invention, the tool shaft is at 90±45 degree angles to the upper extremity of the user.

According to an aspect of some embodiments of the present invention there is provided a laparoscopic tool interface comprising: a frame attachable to an upper extremity of a user; a controller operatively connected to a laparoscopic tool and arranged for manipulation by a hand of a user; and, a mounting on the frame for the laparoscopic tool; and, wherein operating parts for tool position movements of the laparoscopic tool being separated from the controller and wherein a shaft of the laparoscopic tool not being coaxial with the controller.

According to an aspect of some embodiments of the present invention there is provided a laparoscopic tool interface comprising: a frame attachable to an upper extremity of a user; a controller operatively connected to a laparoscopic tool and arranged for manipulation by a hand of a user to effect operation of the laparoscopic tool; and, a mounting on the frame for the laparoscopic tool; and, wherein positioning of the laparoscopic tool is effected by movement of the upper extremity of said user and/or the mounting.

According to some embodiments of the invention, the mounting comprises a joint between the tool shaft of the laparoscopic tool and the frame.

According to some embodiments of the invention, the mounting comprises a pair of jaws for holding the laparoscopic tool; and the frame further comprising a shaft having a worm on its distal end; and a pair of gears in mesh with the worm, and wherein each of the jaws being mounted on a respective spur gear.

According to some embodiments of the invention, there are articulating elements for changing an angle between the shaft of the laparoscopic tool and the upper extremity of the user.

According to some embodiments of the invention, the articulating elements comprise a pair of arc shaped tracks mounted on a distal end of the frame, and means for operatively moving the mounting in relation to the arc shaped tracks to facilitate angular displacement of the laparoscopic tool.

According to some embodiments of the invention, there is an articulated link mechanism for supporting the joint on the frame.

According to some embodiments of the invention, the articulated link mechanism maintains the shaft of the laparoscopic tool in a relative position to the upper extremity of the user.

According to some embodiments of the invention, the articulated link mechanism facilitates changing a relative position of the shaft of the laparoscopic tool with respect to the upper extremity of the user.

According to some embodiments of the invention, the joint facilitates a relaxed position of the laparoscopic device to be at an approximate 90 degree relationship to the upper extremity of the user.

According to some embodiments of the invention, the joint maintaining said shaft of the laparoscopic tool in a relative position to said upper extremity of said user.

According to some embodiments of the invention, the joint maintaining the shaft of the laparoscopic tool in a relative position to the upper extremity of the user even when the upper extremity of said user is moving.

According to some embodiments of the invention, the tool shaft being at an arbitrary angle to the upper extremity of the user.

According to some embodiments of the invention, the tool shaft being at 90±45 degree angles to the upper extremity of the user.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a perspective view, according to an exemplary embodiment of the invention, showing the laparoscopic tool mounted via a passive joint to a frame attached to an upper extremity of the user, and the interface between the surgeon and laparoscopic tool in an electromechanical mode;

FIG. 2 is a perspective view, according to an exemplary embodiment of the invention, showing the frame attached to the surgeon's arm and a chain of adjustable links connecting the frame to the passive joint (the boom adjustment system);

FIG. 3 is a perspective view, according to an exemplary embodiment of the invention, showing an active joint between the interface and the laparoscopic tool;

FIG. 4 is a perspective view, according to an exemplary embodiment of the invention, showing a passive joint between the interface and the laparoscopic tool;

FIGS. 5, 6, 7, 8 and 9 are perspective views, according to an exemplary embodiment of the invention, showing different configurations of holding the laparoscopic tool;

FIG. 10 is a perspective view, according to an exemplary embodiment of the invention, showing palm and finger actuation of the laparoscopic tool;

FIG. 11 is a perspective view, according to an exemplary embodiment of the invention, showing the manner in which the laparoscopic tool is mounted on the interface;

FIG. 12 is a perspective view, according to an exemplary embodiment of the invention, showing forward-back tilting of the laparoscopic tool;

FIG. 13 is a perspective view, according to an exemplary embodiment of the invention, showing right-left tilting of the laparoscopic tool;

FIGS. 14 and 15 are perspective views, according to an exemplary embodiment of the invention, showing degrees of freedom of movement of the laparoscopic tool;

FIG. 16 is a perspective view, according to an exemplary embodiment of the invention, showing angular shifting of the laparoscopic tool;

FIG. 17 is a view, showing a simulation of tool movement from a right-side tool position to a median position;

FIG. 18 is a view showing moments on shoulder muscles due to certain tool movements;

FIG. 19 is a view showing simulation results comparing the changes in palm and elbow height caused by each way of holding the tool;

FIG. 20 is a view showing simulation results comparing the range of movement of various arm joints, caused by each way of holding the tool;

FIG. 21 is a flow chart, according to an exemplary embodiment of the invention, showing a method of using a laparoscopic tool;

FIG. 22 is, according to an exemplary embodiment of the invention, a schematic drawing of a holder for a laparoscopic tool;

FIG. 23A is a side view, according to an exemplary embodiment of the invention, of a handle with a built in controller;

FIG. 23B is a perspective view, according to an exemplary embodiment of the invention, of a handle with a built in controller;

FIGS. 24A-24D are side views, according to an exemplary embodiment of the invention, showing the surgeon moving the laparoscopic tool with respect to an incision (not shown) in the patient body while the passive joint is in a frontal position; and

FIG. 25 is a side view, according to an exemplary embodiment of the invention, showing the surgeon holding the laparoscopic tool with respect to an incision (not shown) in the patient body while the passive joint is in an external position.

### DESCRIPTION OF SPECIFIC EMBODIMENTS OF THE INVENTION

The present invention, in some embodiments thereof, relates to a medical device and, more particularly, but not exclusively, to an interface between the surgeon's body, particularly surgeon's arm, and the laparoscopic tool.

An aspect of some embodiments of the invention relates to separating the functions of positioning of the laparoscopic tool and operating the tool. In an exemplary embodiment of the invention, movement of the upper extremity of the user and/or movement of a joint enable positioning of the tool to a preferred point and at the right orientation. Other body parts of the user, primarily the fingers, effect operational movement of the tool.

Using the existing tools available at the market, the surgeon typically uses the handle of the surgical tool for two functions. First, he has to hold the tool at a desired position. Then, second, he has to operate the tool by pressing/moving/pulling/rotating levers or other mechanisms, as is well known. Often there are conflicts between these functions. In order to hold the tool in the necessary operational position, sometimes the surgeon may need to operate the tool in inconvenient or impossible positions.

The human arm can move/bend in only so many directions. There are certain motions that it cannot make at all, and certain ones that will cause pain or injury to the joints. The shoulder, elbow and wrist are the key joints for movement of the arm. These human joints are not, however, universal joints and they do not have an unlimited range of motion. When, for example, the tool is held along the palm of the surgeon and extends basically parallel to the axis of the arm, generally the surgeon can orient the tool towards the side of the body and make a proper incision and manipulate the tool using small movements in comparison to holding the laparoscopic tool without the passive joint.

When the surgeon has the laparoscopic tool along the palm of his hand and it extends basically parallel to the axis of the arm, he cannot easily work from the top of the patient. This is because his shoulder and wrist do not have a sufficient range of motion to permit manipulating the tool in such an orientation. While the surgeon can work from the side of the patient, once he moves to about 45° (half way to working from the top of the patient), his wrist starts to be unable to move or bend in the necessary direction. Hence, the surgeon is limited in how he may operate the laparoscopic tool when he arranges it along his palm, as is common.

The use of a passive joint for holding the tool allows a much better range of movement of the tool from side to side, whereas conventional holding of the tool allows only half of the range. Further, the amount of work done by the surgeon while using the passive joint is much less than in with a conventional holding.

dH stands for the change in height from the lowest position of the elbow to the highest position of the elbow and is directly connect to the potential energy changes and to the work done by the surgeon's shoulder muscles.

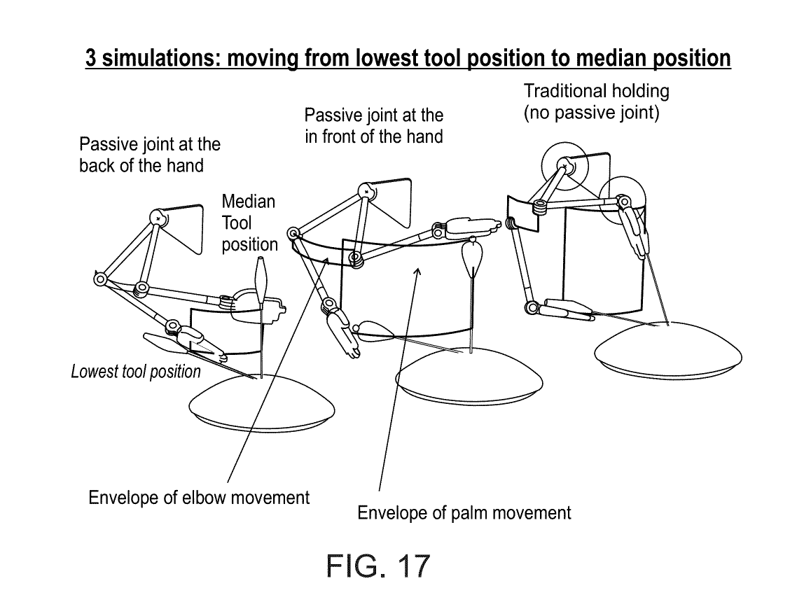

FIG. 17 shows a simulation of tool movement of three types of tools [two have passive joints for holding the tool and one is a conventional mounting] from a right side tool position to a median position.

Similarly, when orientating the tool in other directions, such as distal to the hand or medial to the hand, the surgeon cannot manipulate the tool at certain orientations to the patient, because he does not have the necessary combined range of motion in his shoulder, elbow and/or wrist. As shown in FIG. 17, there is an envelope of elbow movement and an envelope of palm movement. In other words, the elbow and wrist joints can move in only so many directions and not in others. The surgeon is thus limited in how he can use the tool, as, depending on how he holds the tool, certain directions of motion are not physically possible for his arm joints and/or may be physically demanding.

More specifically, the envelopes of movements of the palm and elbow are shown in FIG. 17. The cylindrical envelope has 3 dimensions. The arc represents the angle of movement; and, the height represents the work. The third dimension is the radius of the envelope. The bigger the radius, the bigger the moment the muscles of the surgeon have to balance. This relates to the quality of ergonomics, fatigue of muscles and amount of work exerted by the surgeon.

As shown in FIG. 17, for example, the position of the laparoscopic tool determines the envelope of possible movement. In particular, it illustrates how much movement is required by the elbow to move the tool from the lowest position to the median position. The use of a passive joint for holding the tool increases the size of the envelope of movement of the tool with the smallest amount of elbow movement.

FIG. 17 shows the differences between three ways of holding a surgical tool: a passive joint holding the tool and positioned externally to the palm (or on the back of the palm), a passive joint holding the tool and positioned internally to the palm, and conventional holding of the tool. With conventional holding of the tool, the envelope of movement is relatively small. The use of a passive joint holding the tool and positioned externally to the palm increases the size of the envelope of movement while reducing the amount of work exerted while performing the movement. In addition, this method requires the muscles to produce the smallest moments.

The passive joint holding the tool and positioned on the back of the hand offers the best solution and has the biggest envelop with the smallest elbow movement. This is a benefit. To achieve as big an envelope without a passive joint, it is necessary to significantly increase the elbow movement. The embodiments of the herein disclosed invention are effective because, as explained above, with the passive joint holding the tool, the surgeon can move the tool with much less effort and more ergonomically.

During surgical procedures, the upper arms of the surgeons move. The greater the movement of the upper arm, the greater the physical stress on the upper arm. Therefore, it is advantageous to use the passive hinge for holding the tool as herein disclosed, as it allows maximum movement of the tool, but at the same time minimizes the actual movement of the upper arm and reduces the stress on the upper arm.

The objective is, therefore, to minimize the surgeon's arm movements and to optimize the efficiency of his arm movements. It may be seen that the fewer movements, the better, as it reduces stress on the arm joints, like the elbow.

Sometimes the surgeon needs to hold an instrument that is inserted in the other side of the patient's body. This requires either a long handle for the tool or physical constraints on the arm, and can be problematic for the surgeon. By using the passive joint for holding the tool, the surgeon has the ability to work in his comfort zone and avoid stressful maneuvers, thereby enhancing his control of the tool.

These same issues pertain to the shoulder. If the user or surgeon uses his shoulder to effect movement of the tool while he holds the tool in a conventional way, he might increase the envelope of movement of the tool. By utilizing the passive joint for holding the tool, however, the surgeon does not need to move his shoulder, so that the use of the passive joint leads to more efficient ergonomic work.

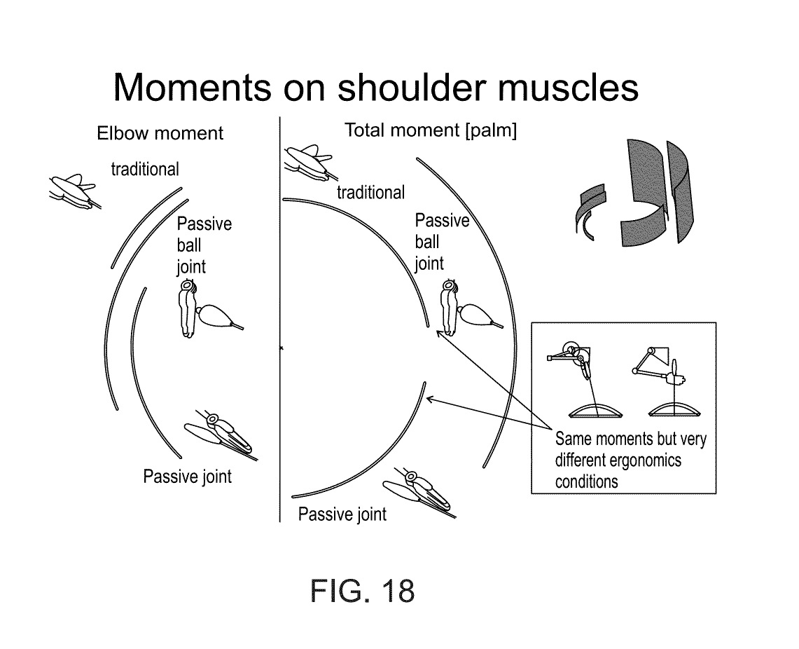

FIG. 18 shows simulation results, showing moments on shoulder muscles caused by different ways of holding the tool while moving the tool from right side to the median plane.

Moment on shoulder or elbow refers to the force applied by the shoulder or elbow muscles to move or stabilize the upper extremity (i.e. the upper arm).

Since the shoulder muscles work against the weight of the tool, the forearm and the arm and the muscles of the elbow work against the weight of the tool and the forearm, the forces applied by the muscles are larger than the moments of the tool itself.

The larger the force, the greater the fatigue and the greater the muscle pain. The radius of the arcs represents the horizontal distance of the center of gravity of the weight from the shoulder or elbow joint.

The bigger the radius, the bigger the moment. To illustrate this concept, consider a heavy suitcase held in a person's hand. People will tend to hold it as near as possible to their body. The reason is that in this position the suitcase is almost beneath the shoulder. Hence there is a minimum radius with a minimum force applied by the muscles. On the other hand, consider the case with the suitcase in a person's hand and the hand is raised to the side. In this position the suitcase is far from the shoulder. Hence there is a greater radius and more muscle force is needed to maintain this position.

This concept pertains equally to laparoscopic surgery. A surgeon generally wants to lessen the moment on his shoulder. By keeping the radius smaller, there is less muscle force required to hold the operating position and less fatigue and discomfit for the surgeon. Use of the herein disclosed joint for holding the tool and interface allow the surgeon to reduce the radius and to use less muscle force. Therefore, there is less fatigue and discomfit.

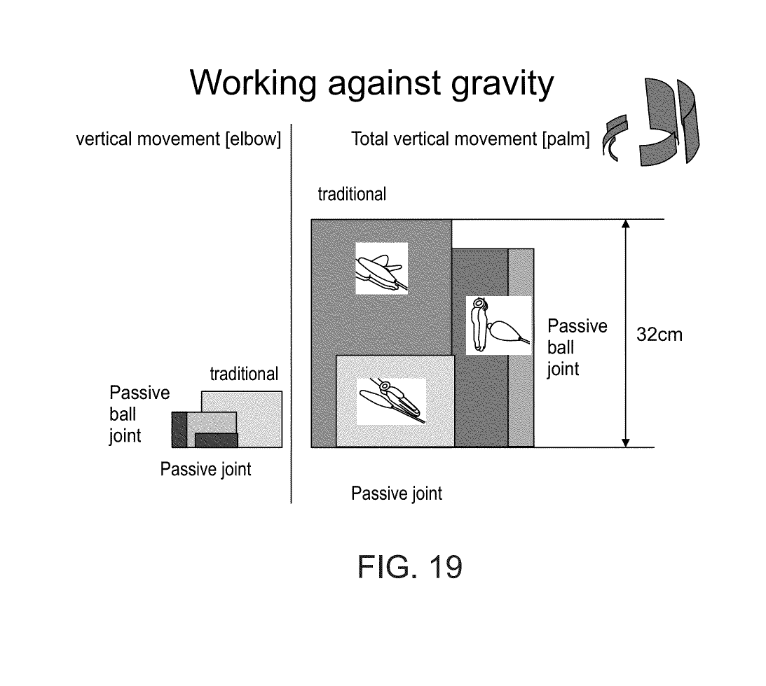

FIG. 19 shows simulation results comparing the changes in palm and elbow height caused by the different ways of holding the tool. It shows the differences in the dH (difference of height which determines the work against gravity). A smaller difference in height translates to less force applied by the surgeon.

If the surgeon needs to work more against gravity in order to obtain the same position, then the surgeon is less effective and the fatigue will come earlier.

Accordingly, by reducing the elbow and palm movement, the surgeon reduces his work against gravity. The herein disclosed joint for holding the tool and interface allows the surgeon to be more efficient and to work less against gravity.

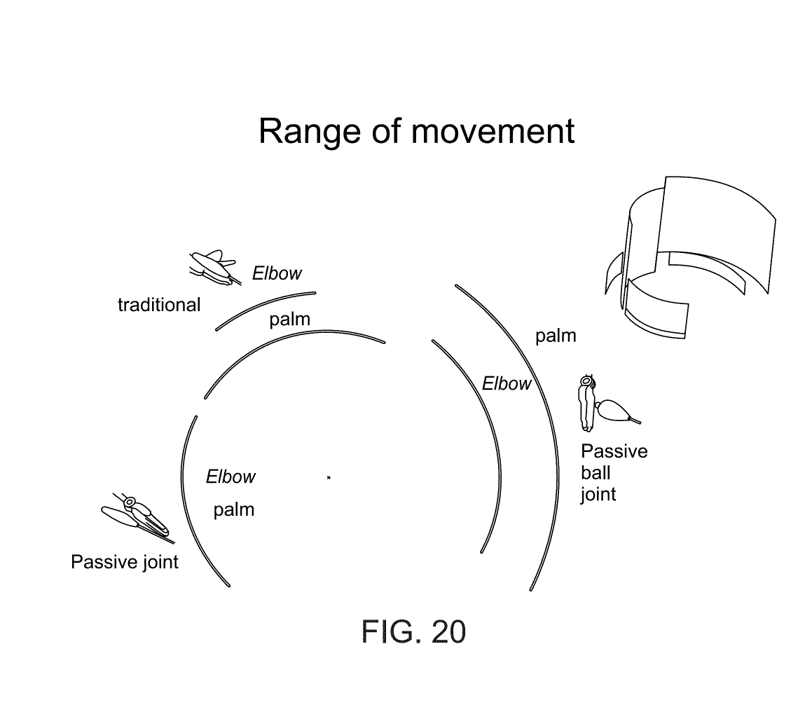

FIG. 20 shows simulation results comparing the range of movement of various arm joints, caused by different ways of holding the tool.

With any laparoscopic tool, the surgeon has a range of movement in which it may be used. As shown in FIG. 20, for example, a traditional or conventional method of holding a laparoscopic tool has a relatively limited range of movement. By comparison, when a passive joint is used to hold the tool, the pattern of the surgeon's movement changes and the potential range of the surgeon's arm increases dramatically. Use of a passive ball joint increases the range of tool movement even more.

Range of movement means the laparoscopic tool total movement. It refers to the surgeon's ability to change the tool angle with respect to the incision in the patient body.

The passive joint for holding the tool allows big changes in tool angle to be achieved while keeping movements of the hand to a minimum. For the passive joint, the arcs are the same, (angle and radius), so they are combined in FIG. 20. As shown, less movement leads to less height changes—the smaller the radius the better.

In summary, the basic concept of the invention is that the functions of positioning of the laparoscopic tool and operating the tool are separated. Movement of the upper extremity (for example the forearm and/or hand) of the user and/or movement of a joint enable positioning of the tool to a preferred position and orientation. The fingers of the user effect operational movement of the tool.

Such separation of the functions of positioning of the laparoscopic tool and operating the tool is advantageous compared to the way a surgeon currently uses laparoscopic tools. At present, the surgeon uses his fingers to hold the tool's positioning and also to operate the tool. This forces the surgeon to work in a non ergonomic manner for long time and to operate the tool with a limitation of finger movement.

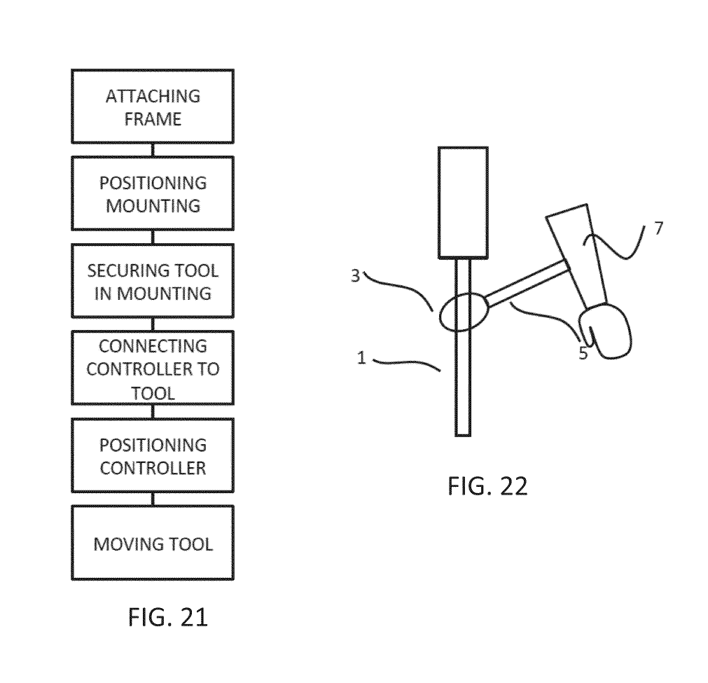

FIG. 21 is a flow chart, showing, for example, a method of using a laparoscopic tool comprising the steps of: attaching a frame to an upper extremity of a user (generally either the forearm or hand); positioning a mounting on the frame for the laparoscopic tool; securing the laparoscopic tool in the mounting; operatively connecting a controller to the laparoscopic tool and arranging the controller for manipulation by a hand of said user; and, initiating movement of the laparoscopic tool by manipulation of the controller and/or by movement of a lower arm of the user.

Movement of the upper extremity (generally the forearm and/or hand) of the user and/or movement of a joint enable positioning of the tool to a preferred point and at the right orientation. The fingers of the user effect operational movement of the tool.

In the disclosure herein the movement of the shoulder is not shown, but sometimes the shoulder may participate in the movement. The herein disclosed method and device for mounting and using a laparoscopic tool contemplates a situation where the surgeon tries to bring the shoulder use to a minimum. A surgeon that operates a few hours a day with his shoulders raised up or pressed down will develop chronic muscle pain and will develop chronic pressures between bones at the Vertebral Column (neck and lower back) and at the shoulder blade. This is why, preferably, the herein laparoscopic tool is typically moved without shoulder movement.

The basic process or manner of using the laparoscopic tool, according to some embodiments of this invention, is to first attach a frame to an upper extremity of a user, such as the forearm or hand. The frame may be of any type, as described hereinafter. It is intended for it to fit easily onto the arm or the hand.

A suitable mounting for the laparoscopic tool is established on the frame. In some embodiments it may be made integral with the frame. Some embodiments may include a distinct mounting affixed in some manner to the frame. The mounting may include a joint, which allows certain ranges of movement of the shaft of the tool. Passive or active joints may be used. Active joints are ones which have some mechanism for moving the shaft of the tool and have the ability of keeping any desired orientation and position of the tool without the need to support the tool with a port. Passive joints are ones which allow free orientation of the tool, but the tool needs to have a support (if not, the tool will move to a resting point determined by gravity).

The shaft of the laparoscopic tool is inserted in the mounting (or joint) and secured. Optionally, the shaft is secured tightly so it does not tilt or rotate within the mounting or joint. In other embodiments, the housing of the tool is secured so the whole shaft may rotate freely.

According to some embodiments of this invention, a controller is operatively connected to the laparoscopic tool and arranged for manipulation by a hand of the surgeon.

In an exemplary embodiment of the invention, movement of the laparoscopic tool is initiated by manipulation of the controller and/or by movement of a lower arm of the user. Since a part of the tool is held in the mounting (or joint) and the mounting remains fixed relative to the arm, movement of the arm necessarily moves the tool. Movement of parts of the tool may be accomplished by using the controller to active an active joint.

In an exemplary embodiment of the invention, the movement of the laparoscopic tool is caused by a combination of movements in the shoulder blade (scapula, clavicle), the movement of the arm bones (Humerus, Radius and Ulna), the palm of the hand, and/or by using the joints of the shoulder, elbow, wrist and fingers of the surgeon.

The use of the joint mounted on the interface allows the surgeon or user to maintain the shaft of the laparoscopic tool in a relative position to the arm of the user.

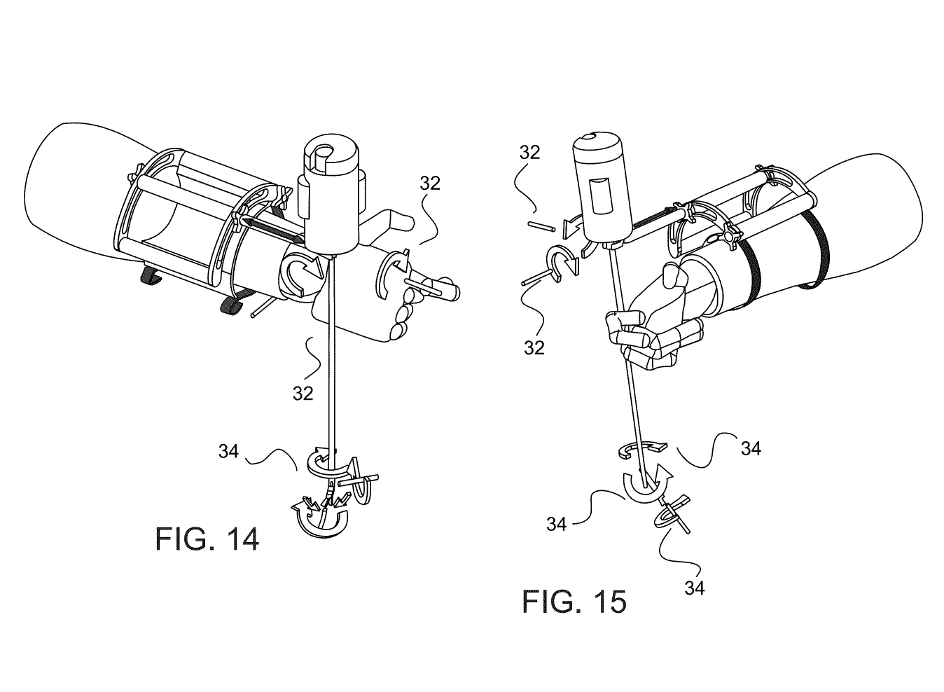

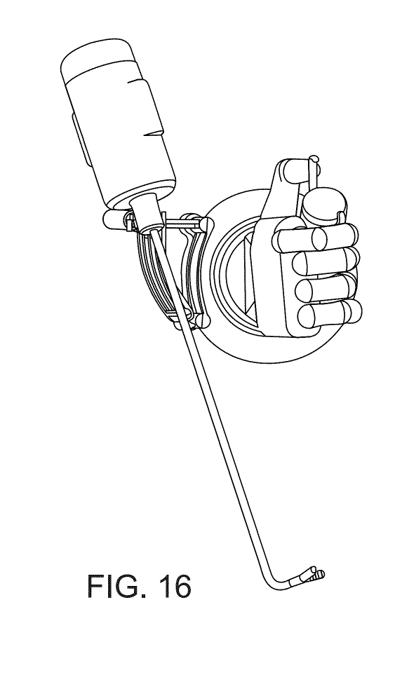

In some embodiments, it is possible to change the relative position of the shaft of the laparoscopic tool with respect to the arm. The joint facilitates this change in relative position. This can involve changing an angle between the shaft of said laparoscopic tool and the arm of the surgeon, as hereinafter described regarding FIG. 16. It could involve tilting the shaft of the laparoscopic tool with respect to the mounting or joint, as hereinafter described regarding FIGS. 14-15.

According to some embodiments herein, the operating parts of the tool are separated from the controlling device and the joint holding the shaft of the tool is separated from the controller. The shaft of the laparoscopic tool is not necessarily coaxial with the arm of the surgeon/user and/or the shaft of the laparoscopic tool is not necessarily coaxial with the controller.

The connection of the laparoscopic device to the frame via a joint allows the movement of the laparoscopic device without having to contort the arm and wrist movements. This is achieved by the shaft of the laparoscopic tool not being coaxial with either the arm of the surgeon or the controller. To accomplish this, the laparoscopic device is connected to the frame via a joint located at any desired position with respect to the frame. If the joint is passive and were to have a free movement, then without having additional support for the tool, the laparoscopic device would move until reaching its gravity resting point, and will stay at an angle to the frame and arm of the user. Therefore, in some embodiments, the device may include a brake mechanism for the passive joint so that the laparoscopic device may be held at a desired angle with respect to the frame.

FIG. 22 is a schematic drawing of one embodiment of a holder for the laparoscopic tool.

In its simplest basic form, as shown schematically in FIG. 22, one embodiment includes a part 1 of the laparoscopic tool held in a joint 3. The joint 3 is held on a supporting member 5 attached in some manner to the arm 7 (or hand) of the surgeon. For utilizing the operating elements of the tool, a controller (not shown) is operatively connected to the laparoscopic tool and arranged for manipulation by the hand of the user.

This construction allows the operating parts for tool position movements of the laparoscopic tool to be separated from the controller. The shaft of the laparoscopic tool is not necessarily coaxial with the arm of the surgeon and/or the shaft of the laparoscopic tool is not necessarily coaxial with said controller.

In an exemplary embodiment of the invention, the functions of positioning of the laparoscopic tool and operating the tool are separated. Movement of the arm of the user and/or movement of a joint enable positioning of the tool to a preferred point and at the right orientation. The fingers of the user effect operational movement of the tool.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

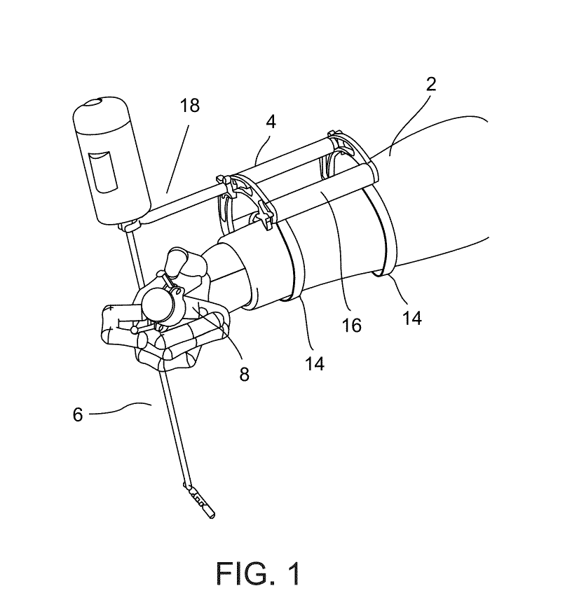

Referring now to the drawings, FIG. 1 is a perspective view, showing the tool mounted via a passive joint to a frame attached to an upper extremity of the user, and the interface between the surgeon and laparoscopic tool is in a manual or electromechanical mode, in accordance with some embodiments of the invention.

FIG. 1 illustrates that, according to some embodiments herein, there is separation between the two functions. In particular, the operating parts of the tool are separated from the controlling device. Both a manual embodiment and an electromechanical embodiment can be effected.

In an exemplary manual embodiment the controller 8 rests in the user's hand. According to some embodiments, a strap may be used to hold the controller 8. Direct mechanical connections 10 extend from the controller to the operative elements of the tool 6. Based on how the controller is manipulated by the user is how the tool will be moved or activated.

The particular design and configuration of the frame or sleeve is not necessarily material to the invention herein. Some embodiments utilize an open frame, whereas others use a closed sleeve. Any suitable material may be used, such as plastic or metal. A soft lining material may be applied to make it more comfortable for the surgeon and to prevent rubbing and irritation on his arm. The frame or sleeve is used to hold the joint and to achieve an operative relationship between the arm and the joint, so that movement of the arm necessarily moves the laparoscopic tool.

For either embodiment, there is a frame (or interface) 4 that is worn over the lower arm 2 (or a part of the lower arm) of the user. The frame may be a simple metal (or rigid plastic) frame. One embodiment utilizes a plurality of rings 14 that encircle the arm 2. For stability, straps 16 connect the rings 14. Extending from the arm is a boom 18 on which the operative controls for moving the laparoscopic tool is mounted. As will hereinafter be explained, the boom may be linearly expandable (telescope) to change the tool's position with respect to the arm of the user.

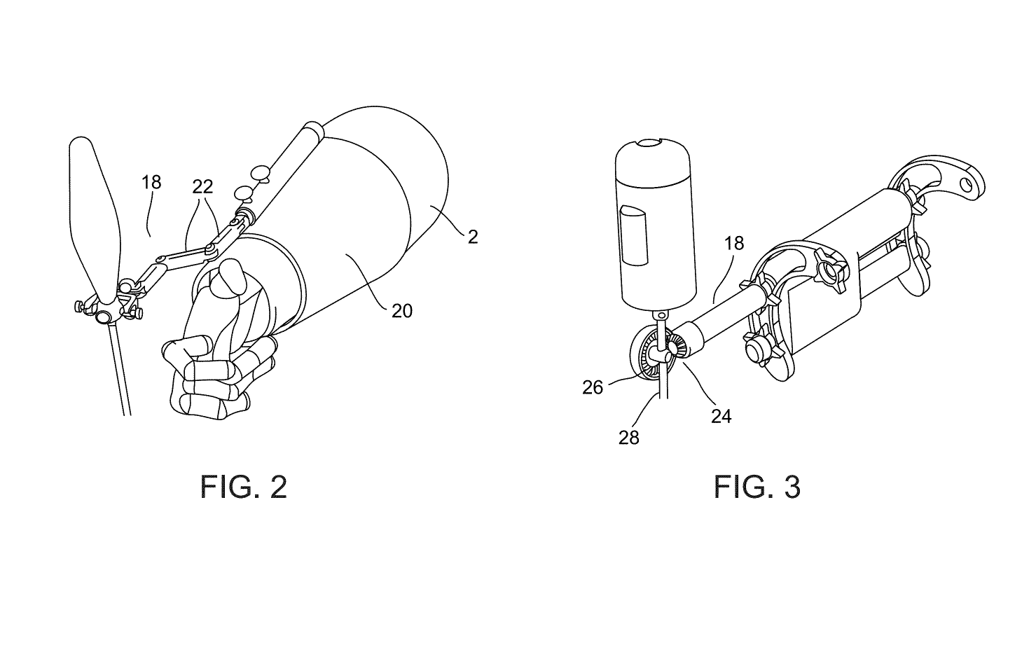

FIG. 2 is a perspective view, showing the frame attached to the surgeon's arm and a chain of adjustable links connecting the frame to the passive joint, in accordance with some embodiments of the invention.

Another embodiment of the frame, as shown in FIG. 2, includes a solid sleeve 20 worn over the lower arm 2 of the user.

Referring to FIG. 2, the boom 18 may include links 22, so that it may be articulated. In this way in addition to telescoping in and out with respect to the length of the arm, it may also be moved from side to side or up and down. By means of the articulated links, many different angles and orientations of the laparoscopic tool can be achieved. This allows the surgeon to move the laparoscopic tool to the position most suitable and efficient for the particular operation being performed.

By means of the articulated links 22, the boom and thus the laparoscopic tool can be moved in many different motion planes and at many different angles. Once a position is determined, the links are optionally locked. This keeps the shaft of the laparoscopic tool in its relative position even when the arm is moved.

In one embodiment, for example, each link has an ear with a central opening on each of its ends. Adjacent links are fitted together so that their ears align. Then, the links are positioned to achieve the correct alignment of the boom, and a pin is inserted through the two openings to hold adjacent links together. A locking nut is used to hold them tight. By unlocking the various pins, the links can be moved to many different angles and orientations, so as to allow the boom to be positioned in a multitude of positions. This enables adjustment of the passive joint holding the laparoscopic tool at any desired spatial position.

At least two basic types of joints may be used for mounting the operative controls of the laparoscopic tool on the boon—a passive joint or an active joint. Essentially, the passive joint does not produce motion but enables free orientation of the tool shaft motion, whereas the active joint does effect motion. In the case of a passive joint, the tool may be supported in an additional point, like the point of incision. The active joint produces moment and forces in the joint. These forces and moments may hold the tool at a desired position without the need of a second support point.

FIG. 3 is a perspective view, showing an active joint connected to the frame, in accordance with some embodiments of the invention.

FIG. 3 shows one possible embodiment of an active joint. On the distal end of the boom 18, a gear 24 is fitted. It meshes with a gear 26 on the shaft 28 of the laparoscopic tool. The motion of the gear train is transferred to the shaft and the operating elements of the tool are thereby employed. The motor for turning the shaft 18 and the gear 26 may be located in the boom and is connected to the gear 24.

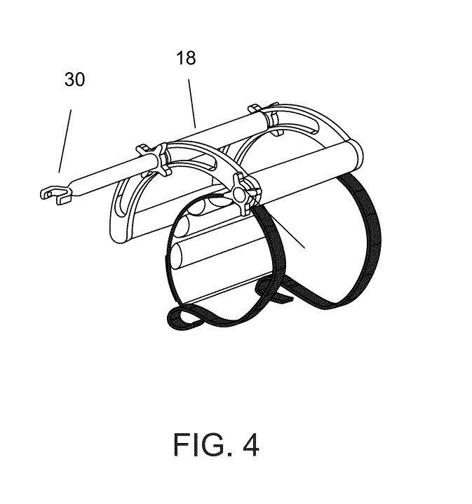

FIG. 4 is a perspective view, showing a passive joint connected to the frame, in accordance with some embodiments of the invention.

A preferred embodiment of a passive joint is shown in FIG. 4 and will be hereinafter described. Generally, it passively holds the shaft of the tool without delivering any moments to the tool. One possible function of a passive joint is to be a first support point for the tool. The combination of the passive joint support point and the second support point at the port (i.e. incision point) enables full control of the orientation of the tool and also enables the surgeon to slide the tool through the incision, inside and outside of the surgical scene.

FIGS. 5-9 are perspective views, showing different configurations of the passive joint positions with respect to the surgeon's arm, in accordance with some embodiments of the invention.

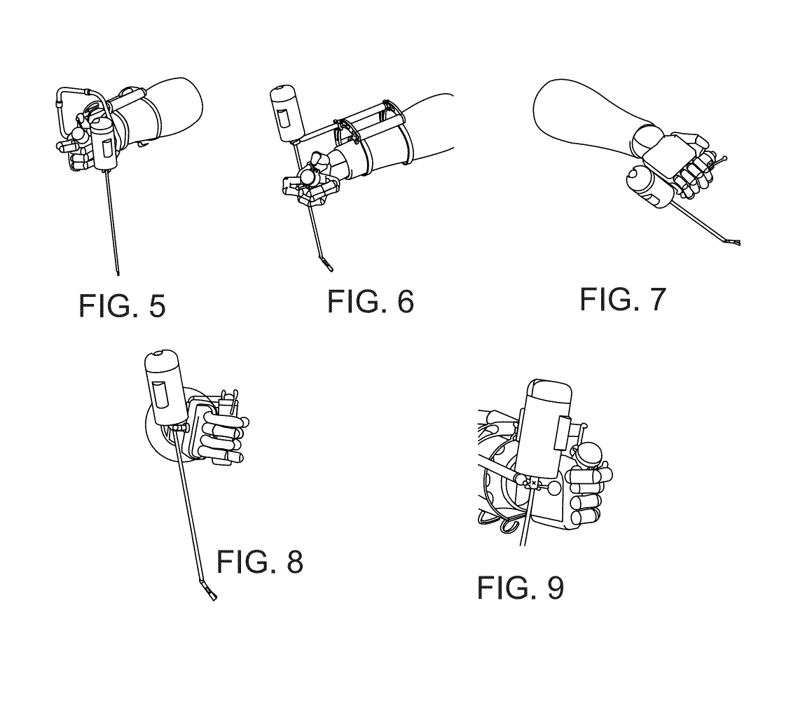

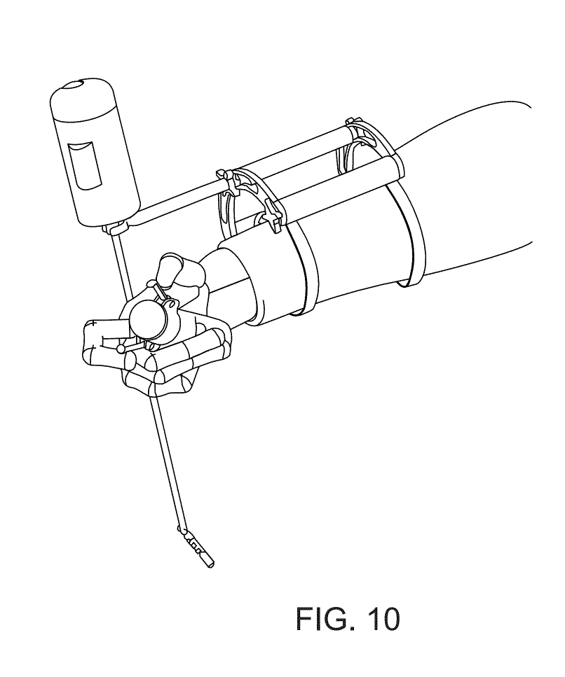

Using a passive joint, the tool may be held medial to the user's hand (FIG. 5), or distal to the user's hand (FIG. 6), or held in the user's hand (FIG. 7), or may be hung on the user's hand (FIG. 10), or may be held by a kinesthetic bridge (FIG. 9).

When a tool is supported by a joint that is held on the surgeon's body, the surgeon may need to adapt to a new way of moving the tool, because he is used to manipulating objects while holding them in their hand. A kinesthetic bridge consists of a small link that connects the joint to a part of the surgeon, in this case his hand. This is not a rigid connection, but a connection that can transfer the location, the movement and orientation of the passive joint to the hand, for example with the sense of friction. As shown in FIG. 9, from the joint a small link rises and touches the surgeon's palm. The surgeon has the ability to increase the force between his palm and the head of the link (the small ball) or, if needed, completely disconnect from the link. This bridge will help the surgeon to feel the passive joint as an extended part of his hand.

FIG. 10 shows a wireless device in the user's hand which transmits a signal to the operative parts of the tool.

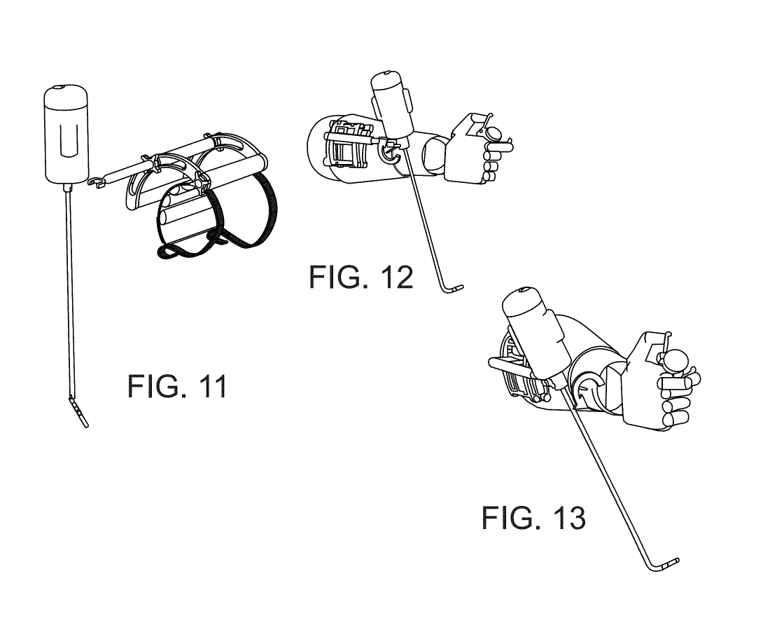

FIG. 11 shows the manner in which the laparoscopic tool is mounted on the passive joint, in accordance with some embodiments of the invention.

FIG. 12 shows forward-back tilting relative to the incision point, using the passive joint of the laparoscopic tool, in accordance with some embodiments of the invention.

FIG. 13 shows right-left tilting relative to the incision point, using the passive joint of the laparoscopic tool, in accordance with some embodiments of the invention.

As shown in FIG. 4, a typical passive joint may include a clamp 30 that fastens around the stem 28 of the tool shaft (or some other part of the tool) and holds it securely. FIG. 10 shows the clamp 30 holding the shaft of the tool (or some other part of the tool), whereas FIG. 11 shows them just before clamping. This type of joint may effect forward and backward tilting of the tool by longitudinal movement of the boom (FIG. 12). Right and left tilting of the tool may be achieved by rotation of the boom (FIG. 13).

Longitudinal movement of the boom is achieved and causes forward and backward tilting by moving the passive joint while supporting the shaft at the incision point.

Side movement of the boom is created and causes right and left tilting. Due to the joint connection, this movement can sometimes be achieved by wrist action (or rotation) of the surgeon, with the boom free to rotate in its housing.

FIGS. 14-15 show degrees of freedom of movement of the laparoscopic tool attached to a passive joint, in accordance with some embodiments of the invention.

Referring to FIGS. 14 and 15, passive degrees of freedom (see arrows 32) are optionally achieved when the movement is the result of movement of the surgeon's arm. Active degrees of freedom are optionally achieved when the movement is the result of the motors in the tool (see arrows 34).

The passive degrees of freedom may allow the surgeon to position, (as a result of his hand movement), the tool at a desired orientation and to move the shaft of the tool through the incision, toward and out of the operating scene. The active degrees of freedom are activated by mechanisms driven by forces or torques and moves parts of the tool for performing the medical procedure.

FIG. 16 shows angular shifting of a first frame part that holds the laparoscopic tool, in accordance with some embodiments of the invention.

To manipulate the operating tool portion of the laparoscopic tool, in accordance with some embodiments of the invention, a controller is optionally used.

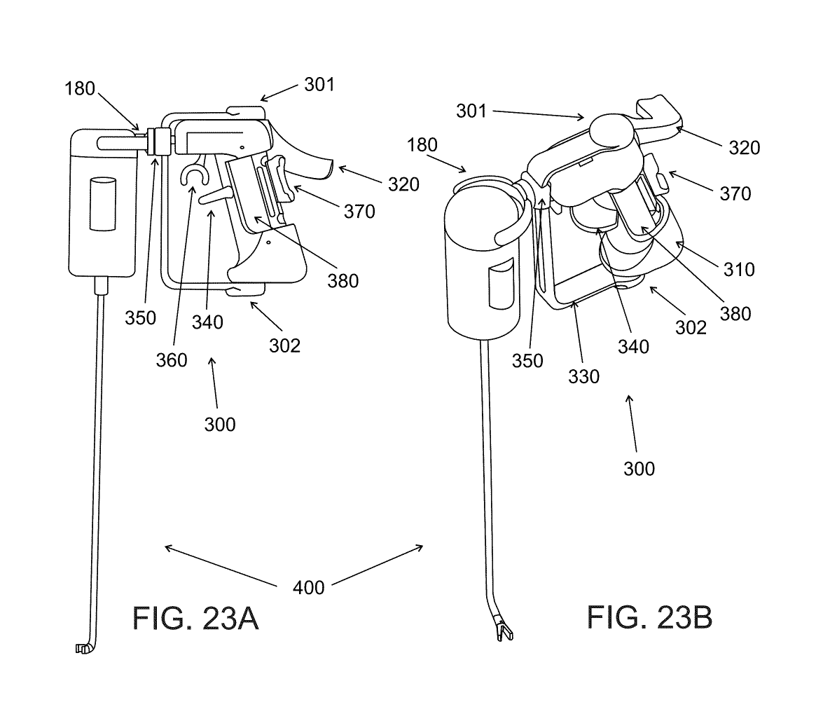

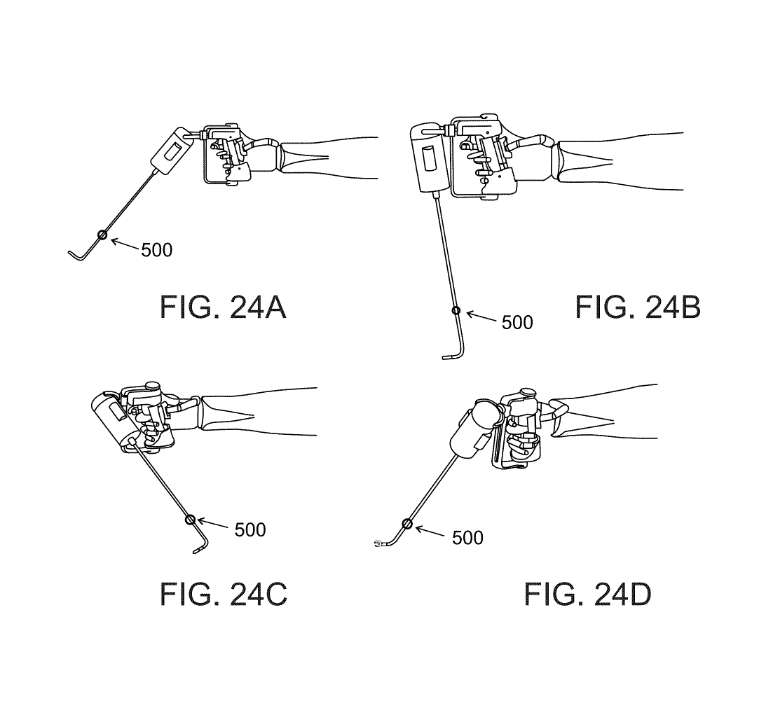

In accordance with some embodiments of the invention, FIGS. 23A and 23B show an exemplary handle 300 with a built in exemplary controller. Knob 370 is preferably operated by the thumb of the surgeon and may slide up and down and rotate. Both movements may be done simultaneously, so the work of the surgeon is continuous. Lever 360 is preferably operated by the index finger of the surgeon and may rotate back and forth and rotate to the side. Both movements may be done simultaneously, so the work of the surgeon is continuous. The laparoscopic tool 400 is attached via a gimbal 180 to a bridge 330. Bridge 330 is connected to the handle 310 via hinges 301 and 302 and may rotate to a preferred position by the surgeon. Bridge 350 allows the surgeon/user to change the height of the gimbal. The arms of bridge 330 may telescopic. Bracket 320 arises to the side of the handle 310 and is used to help the surgeon balance handle 300 on his palm without the need to grasp the handle with a finger. Bracket 340 is also used to assist the surgeon to balance the handle on his fingers without the need to grasp the handle, as shown in FIGS. 24A-D.

FIGS. 24A-D show perspective views of the surgeon moving the tool with respect to an incision in the patient body while the passive joint is in a frontal position.

The advantage of the separation between the position function of the shaft and the means of operating the tool, in accordance with some embodiments of the invention, may be easily seen in FIGS. 24A-D. A wide range of tool movements are achieved by small movements of the surgeon hand. In addition, a change of orientation of the surgeon's hand is not needed even for extreme angles of the tool shaft.

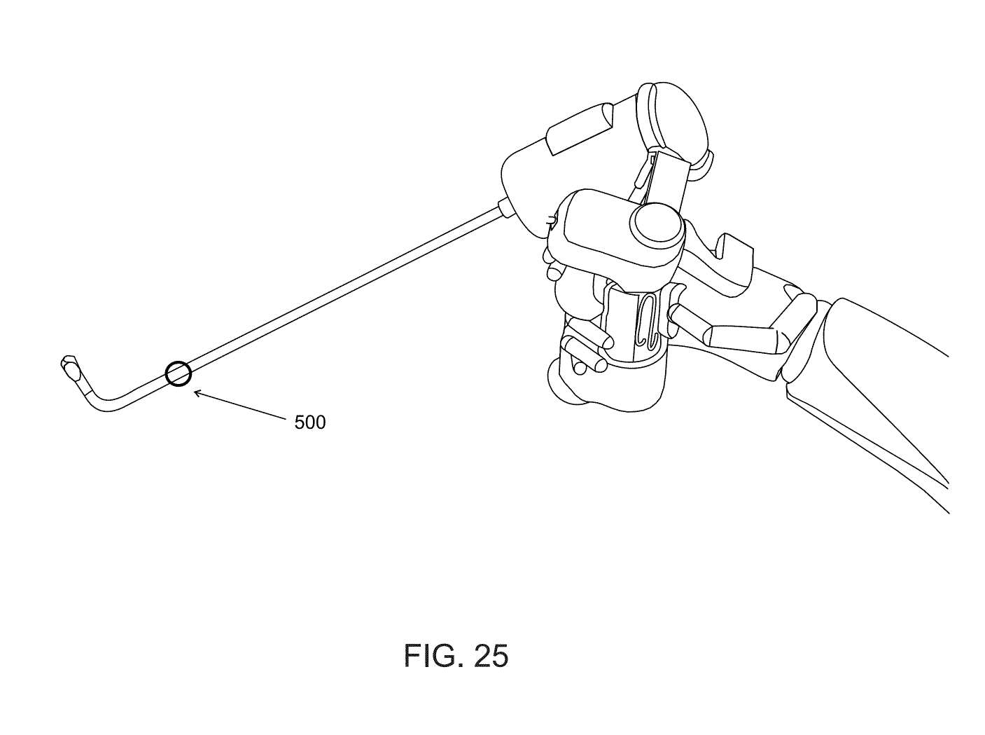

FIG. 25 shows a perspective view of the surgeon holding the tool with respect to an incision in the patient's body while the passive joint is in an external position.

The advantage of the separation between the position function of the shaft and the means of operating the tool, in accordance with some embodiments of the invention, provides for a wide range of tool movement via small movements of the surgeon's hand. In addition, a change of orientation of the surgeon's hand is not needed even for extreme angles of the tool shaft.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A laparoscopic tool interface comprising: a frame attachable to an upper extremity of a user; a controller operatively connected to a laparoscopic tool and arranged for manipulation by a hand of a user; and, a mounting on said frame for said laparoscopic tool; and, wherein operating parts for tool position movements of said laparoscopic tool being separated from said controller and wherein a shaft of said laparoscopic tool not being coaxial with said upper extremity of said user.

2. A laparoscopic tool interface according to claim 1, wherein said mounting comprising: a joint between said tool shaft of said laparoscopic tool and said frame.

3. A laparoscopic tool interface according to claim 1, wherein said mounting comprising: a pair of jaws for holding said laparoscopic tool; and said frame further comprising a shaft having a worm on its distal end; and a pair of gears in mesh with said worm, and wherein each of said jaws being mounted on a respective spur gear.

4. A laparoscopic tool interface according to claim 1, wherein said mounting comprising grasping elements for preventing tilting of laparoscopic tool.

5. A laparoscopic tool interface according to claim 4, wherein said grasping elements for preventing tilting comprises: a pair of jaws for holding said laparoscopic tool; a shaft located in the frame and having a worm on its distal end; and a pair of gears in mesh with said worm, and wherein each of said jaws being mounted on a respective spur gear.

6. A laparoscopic tool interface according to claim 1, wherein said mounting comprising a brake mechanism for preventing rotation of said shaft of said laparoscopic tool.

7. A laparoscopic tool interface according to claim 1, further comprising articulating elements for changing an angle between said shaft of said laparoscopic tool and said upper extremity of said user.

8. A laparoscopic tool interface according to claim 7, wherein said articulating elements comprises a pair of arc shaped tracks mounted on a distal end of said frame, and means for operatively moving said mounting in relation to said arc shaped tracks to facilitate angular displacement of said laparoscopic tool.

9. A laparoscopic tool interface according to claim 2, further comprising an articulated link mechanism for supporting said joint on said frame.

10. A laparoscopic tool interface according to claim 9, wherein said articulated link mechanism maintains said shaft of the laparoscopic tool in a relative position to the arm of the user.

11. A laparoscopic tool interface comprising: a frame attachable to an upper extremity of a user, said frame including a shaft having a worm on its distal end and a pair of gears in mesh with said worm; a controller operatively connected to a laparoscopic tool and arranged for manipulation by a hand of a user to effect operation of said laparoscopic tool; and, a mounting on said frame for said laparoscopic tool, said mounting including a pair of jaws for holding said laparoscopic tool, each of said jaws being mounted on a respective gear of said pair of gears;
wherein positioning of said laparoscopic tool is effected by movement of said upper extremity of said user and/or said mounting.

12. A laparoscopic tool interface according to claim 11, wherein said mounting comprising: a joint between said tool shaft of said laparoscopic tool and said frame.

13. A laparoscopic tool interface according to claim 11, wherein said mounting comprising a brake mechanism for preventing rotation of said shaft of said laparoscopic tool.

14. A laparoscopic tool interface according to claim 11, further comprising articulating elements for changing an angle between said shaft of said laparoscopic tool and said upper extremity of said user.

15. A laparoscopic tool interface according to claim 12, further comprising an articulated link mechanism for supporting said joint on said frame.

16. A method of using a laparoscopic tool comprising the steps of: attaching a frame to an upper extremity of a user; positioning a mounting on said frame for said laparoscopic tool; securing said laparoscopic tool in said mounting thereby allowing a relaxed position of said laparoscopic device to be at an approximate 90 degree relationship to the upper extremity of the user; operatively connecting a controller to said laparoscopic tool and arranging said controller for manipulation by a hand of said user; and, initiating movement of said laparoscopic tool by manipulation of said controller and/or by movement of an upper extremity of said user.

17. A method according to claim 16, wherein said manipulation of said controller by said hand of said user effects operation of said laparoscopic tool; and, positioning of said laparoscopic tool is effected by movement of said upper extremity of said user and/or said mounting.

18. A method according to claim 17, wherein said shaft of the laparoscopic tool is maintained in a relative position to the upper extremity of the user even when said upper extremity of said user is moved.

19. A method according to claim 16, further comprising preventing tilting of said shaft of said laparoscopic tool with respect to said mounting.

20. A method according to claim 16, further comprising preventing rotation of said shaft of said laparoscopic tool in said mounting.
